(19) United States
(12) Patent Application Publication
(10) Pub. No.: US 2008/0208083 A1
(43) Pub. Date: Aug. 28, 2008

(54) System and method to counter material deposition on devices in the urinary tract

(76) Inventors: Bryant Lin, Palo Alto, CA (US); Stewart McCallum, Villanova, PA (US); Tatum Tarin, Redwood City, CA (US); Ross Venook, Burlingame, CA (US)

Correspondence Address: SHAY GLENN LLP, 2755 CAMPUS DRIVE, SUITE 210, SAN MATEO, CA, 94403, US

(21) Appl. No.: 11/796,231

(22) Filed: Apr. 27, 2007

Related U.S. Application Data
(60) Provisional application No. 60/795,493, filed on Apr. 28, 2006.

(51) Int. Cl. A61H 1/00; A61F 7/12

(52) U.S. Cl. 601/2; 607/113

(57) ABSTRACT

The invention relates to a device and method to counteract the deposition of material on devices that dwell in the urinary tract and which include an operably connected motion-inducing actuator. Counteracting material deposition includes impeding or preventing deposition as well as diminishing extant deposition. Deposition is counteracted by moving the device with the motion-inducing actuator; motion may include movement of the device with respect to its position within the urinary tract, as well as deformational or vibrational movement of the device. Embodiments of the device receive energy from a source either by physical or wireless connection. In some embodiments the motion-inducing actuator is integral with the structure of the device, in other embodiments the actuator is a separate structure. In still other embodiments, the device receives energy directly, without intervention of an actuator. In some of these latter embodiments, the device may include an energy focusing element.

### CROSS REFERENCE TO RELATED APPLICATIONS

This application further claims priority under 35 U.S.C. § 119 to U.S. Ser. No. 60/795,493 of Lin and McCallum, filed on Apr. 28, 2006, entitled “Method to prevent material deposition and encrustation on devices in the urinary tract”, the disclosure of which is incorporated herein by this reference.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

The invention is in the field of medical devices related to urogenital health, more particularly to devices that dwell in the urinary tract.

### BACKGROUND OF THE INVENTION

The urinary tract extends from the collecting tubules in the kidney where urine is excreted from the nephrons to the meatus of the urethra where the urine exits the body. Many devices in contact with the urinary tract are subject to a problematic deposition of biomaterials from the urinary tract environment. The devices can readily become a nidus for stone formation or encrustation from soluble elements that precipitate and accumulate on the surface of such devices. Such accumulated deposition can occur on the inner luminal surface of devices, even to the extent of occluding them, and on the outer surface, creating pain and complications, such as the inability to remove the device. Formation of such encrustration is very common, if not nearly inevitable with sufficient time, on devices situated in the urinary tract. Similarly, although with less frequency, clots or thrombi can form around devices in the urinary tract.

A variety of indwelling medical devices are used to either treat conditions or to functionally substitute for portions of the urinary tract along its entire length, from the kidney to the urethral opening. Common urinary devices in men and women include ureteral stents for treating obstruction of the ureter, ureteric fistulas, and gaps or defects caused by trauma or other conditions, and incontinence devices for treating the lack of control of micturition and incontinence. Foley catheters are used for dealing variously with controlling continence, urinary tract obstruction, and sphincter restriction of flow following anesthesia. In men, devices further include urethral catheters for treating benign or cancerous prostate growth, urethral strictures, and incontinence, and penile prosthetics for treating erectile dysfunction. In some cases, these devices are inserted for relatively short duration, but in the case of indwelling stents, they are typically used for much longer periods of time, and are typically changed every three to six months to prevent clinically problematic encrustation.

Long term urethral catherization for intractable urinary incontinence or retention is particularly common among patients in chronic care facilities, and a large portion of these patients experience catheter blockage. Such blockage generally can cause pain, and incontinence to due urine bypassing the catheter, and is associated with uriny tract infections. Encrustation even without blockage can cause pain, and can severely complicate the process of changing cathethers. The removal of an indwelling stent that has become encrusted can turn a normally minor procedure into a highly involved and difficult one, placeing the patient at risk of unexpected complications and also the need for costly and time consuming surgical procedures. The clinical significance of encrusted stents is serious and well known, as reported for example, by Singh et al. (Severely encrusted polyurethane ureteral stents: management and analysis of potential risk factors. Urology 58 (4) 526-531, 2001). Bultitude et al. (Management of encrusted ureteral stents impacted in upper tract. Urology 62 (4) 622-626, 2003.), and Park et al. (Clinical features determining the fate of a long-term, indwelling, forgotten double J stents. Urol. Research 32: 416420, 2004).

Bacterial infection can be associated with encrustation either as a contributing cause or as a complication. Several patents have described approaches to mitigating encrustation by way of incorporating coatings on indwelling urinary devices that provide a level of bacterial resistance or resistance to adhesion of foreign materials, such as U.S. Pat. No. 5,328,954, U.S. Pat. No. 5,877,243, and U.S. Pat. No. 5,935,094. Encrustation and the deposition of materials on devices in the urinary, however, involves the basic processes that underlie urinary stone formation even without the causes or complications associated with infection. The problem of deposition of material on indwelling urinary tract devices remains very prevalent, and is in need of new and more effective preventative and interventional approaches.

### SUMMARY OF INVENTION

The invention relates to material deposition-countering urinary tract device that includes a structure adapted to dwell at least partially in the urinary tract and an actuator operably coupled to the structure such that actuator activation induces motion of the structure. Embodiments of these indwelling device may include any of a ureteral stent, an upper urinary tract stent, a lower urinary tract stent, a catheter, a urethral prosthesis, a urethral insert, an artificial urinary sphincter, a nephrostomy tube, a suprapubic tube, a urethral plug, or any structure adapted to dwell within the urinary tract. The device embodiments may dwell in any portion of the urinary tract, including any one or more portions of a kidney, a ureter, a bladder, or a urethra. Embodiments of the device include a surface with an area, and the induced motion includes a transient reconfiguration of the surface area.

The actuator included in embodiments of the device may include any of a motor, a mechanical transducer, a piezoelectric element, a shape memory polymer, an electroactive polymer, a shape memory alloy, a miniature actuator, an electromechanical actuator, or an electromagnetic actuator. Embodiments of the device may be configured to receive energy conveyed by a physical connection, or they may be configured to receive wireless energy. In some embodiments, the device is further configured to focus received wireless energy. In particular embodiments the received energy is ultrasonic energy.

In some embodiments, the motion-inducing actuator and the structure of the device are structurally integrated, in other embodiments, though being operably connected, they are not structurally integrated.

In some embodiments, the motion-inducing actuator includes a mechanical transducer powered by a physiological process. In some of these embodiments, the physiological process includes urine flow, and the mechanical transducer includes a fluid-flow energy transducer in the urine flow path.

In some embodiments, the device further includes an energy source to power the motion-inducing actuator. In some of these embodiments, the energy source is physically connected to the actuator, for example, an energy storage device, such as a battery or a capacitor. In other embodiments, the device is configured to deliver energy wirelessly. In some of these embodiments, the wireless energy source may be any of an inductive coil, a microwave emitter, a radiofrequency emitter, or a sonic emitter. Some embodiments of the wireless energy source are located external to the body, some of these may by attached to the body, or held in an article attached to the body, or worn by the patient. In other embodiments, the energy source is unattached to the patient. In still other embodiments, the energy source is implanted in the body.

Some embodiments of the device further include a wireless energy receiver configured to convey energy to the motion-inducing actuator. Various of these embodiments may include any of an antenna, a sonic transducer, or an electromagnetic-mechanical transducer.

Embodiments of the device may be subject to material deposition on any of an external surface of the device or an internal surface of the device, the device countering such material. In some embodiments, the deposition may include a precipitate of at least one solute from urine. Such solutes may include any one or more of calcium oxalate monohydrate, calcium oxalate dihydrate, calcium phosphate, magnesium ammonium phospate, struvite, hydroxyapatite, carbonate apatite, brushite, cystine, indinavir, triamterene or uric acid. In other embodiments, the deposition is of thrombotic origin. In still other embodiments, the deposition may include cellular material, such as any of whole cells, cell fragments, or cell-derived debris. In still other embodiments, the deposition may include a biologically-derived film or biofilm, such as a film derived from the patient or from an organism that has infected the patient or is naturally resident in the patient. In some instances the deposits may include medicines taken by the patient or metabolic products thereof that precipitate out in the urine.

The invention further relates to material deposition-countering urinary tract system that includes a device adapted to dwell at least partially in the urinary tract and an energy source adapted to provide energy to move the device. Embodiments of the device may include any of a ureteral stent, an upper urinary tract stent, a lower urinary tract stent, a catheter, a urethral prosthesis, a urethral insert, an artificial urinary sphincter, a nephrostomy tube, a suprapubic tube, a urethral plug, or any structure adapted to dwell within the urinary tract. The device may dwell in any portion of the urinary tract, including any one or more portions of a kidney, a ureter, a bladder, or a urethra. Embodiments of the device include a surface with an area, and in some embodiments, the induced motion induces a transient reconfiguration of the surface area.

In some embodiments of the system, the device is adapted to focus received wireless energy, such as, for example, ultrasonic energy. In some of these embodiments, the adaptation includes an energy focusing element that moves in response to the energy, the element configured to impart movement to the device as a whole. In some embodiments, the energy focusing element is attached to the device, in other embodiments, energy focusing element is integral to the device. In still other embodiments, the adaptation includes the device having a composition with an elastic modulus different from that of a conventional device. In other embodiments, the adaptation includes the device having a physical form that supports a resonant standing wave.

In some embodiments of the system, the energy source is configured to provide energy by physical connection; in other embodiments the the energy source is configured to provide energy wirelessly. Embodiments of a wireless energy source include any of an inductive coil, a microwave emitter, a radiofrequency emitter, or an ultrasonic emitter.

In some embodiments of the system, the energy source may be included internally within the body; in other embodiments, the may be is external to the body. In embodiments, where the energy source is disposed external to the body, the energy source may be attached to the patient, including being held in an article attached to the body, or worn by the patient. In other embodiments, the energy source is unattached to the patient. In still other embodiments, the energy source is implanted in the body.

Embodiments of the system may be subject to material deposition on any of an external surface of the device or an internal surface of the device, the device countering such deposition of material. In some embodiments, the deposition may include a precipitate of at least one solute from urine. Such solutes may include any one or more of calcium oxalate monohydrate, calcium oxalate dihydrate, calcium phosphate, magnesium ammonium phospate, struvite, hydroxyapatite, carbonate apatite, brushite, cystine, indinavir, triamterene, or uric acid. In other embodiments, the deposition is of thrombotic origin. In still other embodiments, the deposition may include cellular material, such as any of whole cells, cell fragments, or cell-derived debris. In still other embodiments, the deposition may include a biologically-derived film or biofilm, such as a film derived from the patient or from an organism that has infected the patient or is naturally resident in the patient.

The invention further relates to a method of countering material deposition on a urinary tract system that includes a device adapted to dwell at least partially in the urinary tract and an energy source adapted to provide energy to move the device, the method includes moving the device. In some embodiments of the system, the system further includes an actuator operably coupled to the structure such that actuator activation induces motion of the device.

In some embodiments of the method, the energy source conveys energy to the motion-inducing actuator by a physical connection; in other embodiments, the energy source may configured to provide energy wirelessly.

In some embodiments, the energy source in configured to convey energy to the motion-inducing actuator. In other embodiments, the device further comprises an energy focusing element that moves in response to receiving energy, the element configured to then impart such movement to the device as a whole.

Embodiments of the method of moving the device include moving it sufficiently to counter the presence of material on the device. Countering the deposition may include any of impeding material deposition, preventing material deposition, diminishing an extant deposition of material, or clearing a device occluded by deposited material such that urine may flow through the structure.

Embodiments of the method of moving the device include moving the device translationally with resonance along any of linear or rotational degree of freedom, moving the device translationally without resonance along any of linear or rotational degree of freedom, or moving the device deformationally with modes of any order, along any of linear or rotational degrees of freedom, or any combination of such types of translational and deformational movement.

Embodiments of the method of moving the device include moving the device sufficiently to counter the presence of material deposition comprises moving the device for a sufficient amount of time. Moving for a sufficient amount of time may include any of sufficient total amount of time per day, a sufficient total amount of time per week, or a sufficient amount of time per month, or a sufficient period of time during the residence of the device. In some embodiments of the method, moving the device occurs episodically, in which instance a sufficient period of time may include moving the device for a sufficient amount of time per episode.

In other embodiments of the method, moving the device sufficiently to counter the presence of material deposition includes moving the device with sufficient power. In some embodiments, moving with sufficient power may include moving the device with a sweep of a range of power. In other embodiments, moving the device sufficiently to counter the presence of material deposition includes moving the device at an effective vibrational frequency. In some of these embodiments, moving at an effective vibrational frequency includes moving the device with a sweep through a range of frequencies.

The invention still further relates to a material deposition-countering bodily indwelling system that includes a device adapted to dwell at least partially in a space in the body and an energy source adapted to provide energy to move the device. In some embodiments, the system further includes an actuator operably coupled to the structure such that actuator activation induces motion of the device. In other embodiments, the device is configured with an adapative feature that focuses received energy, or includes an incorporated or attached element that focuses received energy. In some embodiments received energy causes heating in addition to or instead of movement. In some embodiments, the space in the body comprises a lumen, such as, by way of example, any of an auditory canal, a blood vessel, a heart, a bile duct, a salivary gland, a parotid gland, a sinus, or a lacrimal duct, or a pulmonary lumen. In other embodiments, the space in the body includes a cavity, such as, for example the pleural cavity or the peritoneal cavity. In still other embodiments, the space in the body includes the alimentary canal. In still further embodiments, the space in the body comprises an interstitial space, as may occur in any tissue or organ in the body. The inventions further relates to a method of using the material deposition-countering bodily indwelling system, the method including moving the device. Moving the device may include any method summarized above, as applied to an indwelling urinary tract device and system.

### BRIEF DESCRIPTION OF THE DRAWINGS

FIGS. 1A-1E are block diagrams of various embodiments of a deposition-countering urinary tract device. FIG. 1A depicts an embodiment of a urinary tract device in which the actuator and the device are structurally integrated, and receive power from a physically connected energy source. FIG. 1B depicts an embodiment of a device that is structurally integrated with an actuator, the actuator receiving power through an energy receiver, the power being wirelessly transmitted to the receiver from an energy source. FIG. 1C depicts an embodiment of a device in which an actuator and a device are structurally separate but attached to each other, the actuator receiving power from a physically connected energy source. FIG. 1D depicts an embodiment in which an actuator and a device are structurally separate but attached to each other, the actuator receiving power through an energy receiver that is wirelessly transmitted to the receiver from energy source. FIG. 1E depicts an embodiment of a device that directly receives energy wirelessly from an energy source.

FIG. 2A shows a prior art indwelling device within a portion of a urinary tract; FIG. 2B shows a cross section of the devise within the tract with material deposition on the exterior surface of the device. FIG. 2C shows a cross section of the device with material deposition on the interior surface of the device.

FIG. 3A shows an indwelling material deposition-countering device within a portion of a urinary tract; FIG. 3B shows a cross section of the devise within the tract.

FIG. 4A shows an exposed view of a portion of a ureter with a perspective view of a portion of an indwelling device with a motion-inducing actuator in the form of a longitudinal strip in an unpowered state.

FIG. 4B shows an exposed view of a portion of a ureter with a perspective view of a portion of the indwelling device with a motion-inducing actuator in the form of a longitudinal strip of FIG. 4A being powered by an energy source.

FIG. 5A shows an exposed view of a portion of a ureter with a perspective view of a portion of an indwelling device with a motion-inducing actuator in the form of a series of circumferential rings in an unpowered state.

FIG. 5B shows an exposed view of a portion of a ureter with a perspective view of a portion of the indwelling device with a motion-inducing actuator in the form a series of circumferential rings of FIG. 5A being powered by an energy source.

FIG. 6A shows a perspective view of a portion of an indwelling device with a motion-inducing actuator in the form of a longitudinal strip in an unpowered state.

FIG. 6B shows a perspective view of a portion of the indwelling device with a motion-inducing actuator in the form of a longitudinal strip of FIG. 6A being powered by an energy source, further depicting vibratory motion induced by the motion-inducing actuator.

FIG. 7 shows a perspective view of a portion of an indwelling device with a motion-inducing actuator in the form of a series of circumferential rings being powered by an energy source, further depicting vibratory motion induced by the motion-inducing actuator

FIG. 8 shows a perspective view of a portion of an indwelling device with a motion-inducing actuator in the form of circumferential spiral.

FIG. 9 shows a perspective view of a portion of an indwelling device with a motion-inducing actuator in the form of a series of longitudinal strips.

FIG. 10 shows a perspective view of a portion of an indwelling device with a motion-inducing actuator in the form of a lattice.

FIG. 11 shows a perspective view of a portion of an indwelling device with a motion-inducing actuator in the form of multiple isolated loci.

FIG. 12A and 12B show a perspective view of an indwelling device with a motion-inducing actuator in the form of a longitudinally configured wave, motion being induced in the device and two states being shown. FIG. 12A depicts a portion of the device in a linearly contracted state. FIG. 12B depicts the portion of the device while in a linearly expanded state.

FIGS. 13A-13D depict a device in the form of a double J stent with various embodiments of a motion-inducing actuator: 13A shows a discontinuous series of longitudinal strips, 13B shows a continuous fully encompassing motion-inducing actuator, 13C shows a continuous longitudinal strip, and 13D shows a continuous spiral strip.

FIG. 14 is an exposed view of a portion of an indwelling device with a urine flow energy transducer in the form of a propeller with an eccentric counterweight.

FIG. 15 is an exposed view of a portion of an indwelling device with a urine flow energy transducer in the form of a wheel operating against a flipper.

FIG. 16 is an exposed view of a portion of an indwelling device with a urine flow energy transducer in the form of vibrating flaps.

FIG. 17 is a perspective view of a device with solid body device that includes integrated antenna and motion actuating elements in the form of woven wire elements.

FIG. 18 is a perspective and partially cut-away view a portion of a device with an antenna and motion-actuating elements integrated into the device.

FIG. 19 is a perspective view of a device in the form of a bare metal wire stent, with antenna and motion-inducing actuator elements integrated into the woven wire.

FIG. 20 shows a face-on flattened planar view of a portion of a wall of a device with solid reference lines depicting the surface area in a neutral state and dotted reference lines depicting a stop-action view of the surface area when the device is vibrating due to motion actuation, and the reference lines depicting the distortion of the surface area, with some sectors expanded, and others contracted.

FIG. 21 shows an embodiment of a device in the form of a Foley catheter, a partially indwelling urinary tract device, with a motion-inducing actuator integrated into the device.

FIG. 22 shows an embodiment of a device in the form of a Foley catheter, a partially indwelling urinary tract device, with a motion-inducing actuator attached to the device.

FIGS. 23A-C shows an embodiment of a device in the form of a Foley catheter, a partially indwelling urinary tract device, with a motion-inducing actuator in the form of a sleeve fitted over the balloon end of the catheter. FIG. 23A shows the balloon portion of the Foley deflated, the actuator sleeve prior to be fitted over the balloon, FIG. 23B shows the sleeve fitted over the balloon portion, and FIG. 23C shows the sleeve over the balloon portion after the balloon has been inflated.

FIG. 24 shows an exposed side view of a device with a wall that is adapted to enhance resonance upon receiving energy.

FIG. 25 depicts a side view of a portion of a device that incorporates an energy-focusing element.

FIG. 26 shows a patient with an indwelling device in the form of a double J stent, the device being powered by an external energy source.

FIG. 27 shows a patient with an indwelling device in the form of a double J stent, the device being powered by a handheld external energy source.

### DETAILED DESCRIPTION

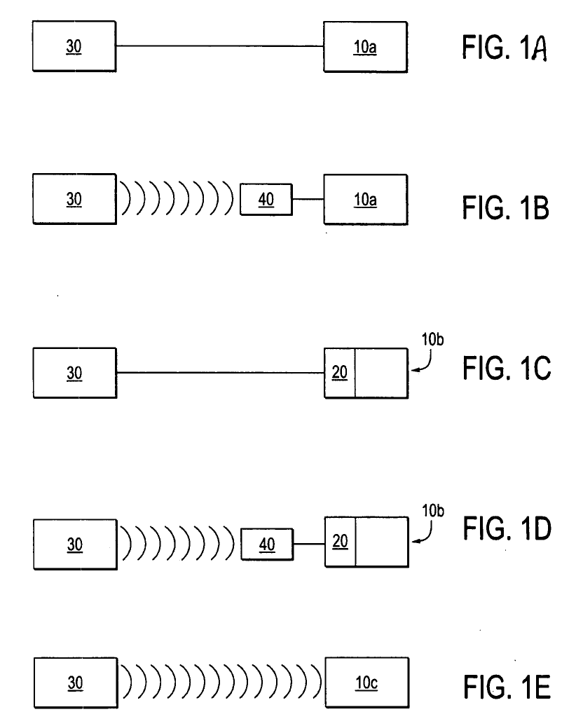

FIGS. 1A-1E depict various embodiments of the inventive urinary tract device that are capable of countering the deposition of material from the urinary tract within which it dwells. The countering of material deposition includes impeding or resisting deposition, preventing deposition, and it further includes the disruption and detachment of material that may already be deposited on the surface of the device. In some embodiments of the device, countering the deposition of material occurs through movement, either by movement within and with respect to the position of the device within the urinary tract (translational movement) or by vibration of the device (deformational movement), or by a combination of translational and deformational movement.

In broad aspect, the invention relates to a device and a method to operate the device wherein an indwelling device moves in response to an input of energy, and by such movement the device counters the deposition of material as described above. In some embodiments, the movement is mediated by an operably connected movement-inducing actuator included with the device that transduces the energy input into the movement. Embodiments such as these are schematically represented by FIGS. 1A-1D.

In other embodiments, schematically represented by FIG. 1E, a device is moved by an input of energy directly, without participation of an energy-transducing actuator. These latter embodiments, when joined with an energy source or emitter, may comprise a system that can include several variations. In some variations, the urinary tract device is conventional, and the system includes an energy emitter that moves the conventional device translationally, deformationally, with a combination of translation and deformation, as will described in further detail below. In other embodiments, the device is not conventional, but rather is particularly adapted to receive energy and efficiently transduce it into movement. In some of these embodiments, the adaptation includes particulars of the composition of the materials of the device, or the shape of the device, which underlie the efficient transduction of ultrasound into movement, for example. In other embodiments, the adaptation may include a distinct energy-focusing or energy-targeted element attached or included within the device. This energy-focusing element is distinct from an actuator that actively transduces receives energy from one form to another, thereby inducing motion (per embodiments depicted in FIGS. 1A-1D). The energy focusing element may be adapted to resonate with high efficiency in response to impinging ultrasonic energy, for example. The resonance of such an element, by virtue of being integrated with or attached to a device, provides resonant movement to the device as a whole. Such an element may be applied to a device otherwise conventional, or it could be applied to a device which itself, is also particularly adapted by composition or form to move in response to ultrasound, for example.

With regard to a device, as just described in the context of an embodiment depicted by FIG. 1E, where the device is particularly adapted by composition or form to resonate efficiently, such adaptive features of composition or form may also be included in any device represented by FIGS. 1A-1D. The basic aspect that distinguishes the embodiment of FIG. 1E from the embodiments of FIGS. 1A-D is the absence of an actuator. The same features of FIG. 1E that serve to optimize the movement response to impinging ultrasound energy, may also be beneficially applied to embodiments of the device that include an actuator.

Turning now to the various embodiments of the device that include a motion-inducing actuator, such actuator is operably connected with the structure of the device, and causes, on activation, movement of the device. The nature of the operable connection may vary, and does vary in accordance with the nature of the physical relationship between the actuator and the device. In some embodiments, the actuator and the device are structurally integrated, in some embodiments the actuator and the device, though operably connected are structurally separate or separable. Actuators require power or energy input to impart movement to the device; accordingly, energy may be conveyed to the actuator from an energy source by physical connection, by wireless connection, or by any other suitable mode or combination of modes. Typically. actuators that receive power by wireless means utilize an energy receiver such as an antenna to receive power from the source and convey it to the actuator. In some embodiments, where the device acts as its own actuator, the actuator-device may receive the energy itself also, without the intervention of a separate energy receiver.

Turning now with reference to the block diagrams of FIGS. 1A-1E and the various embodiments of a deposition-countering urinary tract device, FIG. 1A depicts an embodiment of a urinary tract device 10a in which the actuator and the device are structurally integrated, and receive power from a physically connected energy source 30. FIG. 1B depicts an embodiment of a device 10a that is structurally integrated with an actuator, the actuator receiving power through an energy receiver 40, the power being wirelessly transmitted to the receiver 40 from energy source 30. FIG. 1C depicts an embodiment of a device 10b in which an actuator 20 and a device 10b are structurally separate but attached to each other, the actuator 20 receiving power from a physically connected energy source 30. FIG. 1D depicts an embodiment in which an actuator 20 and a device 10b are structurally separate but attached to each other, the actuator 20 receiving power through an energy receiver 40 that is wirelessly transmitted to the receiver 40 from energy source 30. Finally, FIG. 1E depicts an embodiment of a device 10c (as detailed above in a comparative way with respect to embodiments of the preceding FIGS. 1A-1D) that lacks a distinct actuator or energy receiver, receiving energy wirelessly from energy source 30.

In the various embodiments described below, indwelling devices will generally be designated as device 10, this designation can refer to any device, whether it has a movement-inducing actuator integrally incorporated therein, or whether it is has a motion-inducing actuator operably that is operably connected to the structure of the device, but structurally distinct therefore, of a device that lacks a motion-inducing actuator. In cases where the distinction is needed for an understanding of the invention, designation of device 10a will refer to devices with an integrated actuator, device 10b will refer to devices with a separate actuator, and device 10c will refer to devices that lack a distinct actuator and/or own energy receiver.

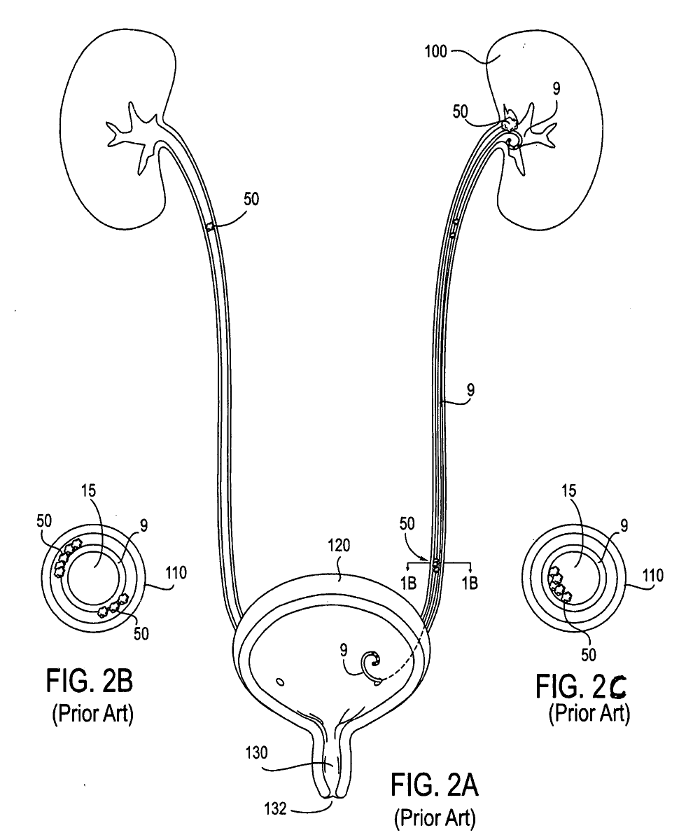

Various illustrative examples of embodiments of the invention will now be provided; it may be understood that variations on these embodiments may exist without departing from the basic elements described, including a device, a motion-inducing actuator, an energy receiver, and an energy source. As described, these elements may be clearly understood in terms of their function and functional relationships, while the physical form and relative degree of structural integration, connection, or quality of single structure combining various functions may vary. In FIGS. 2-27 that follow, various specific embodiments are shown, as described below.

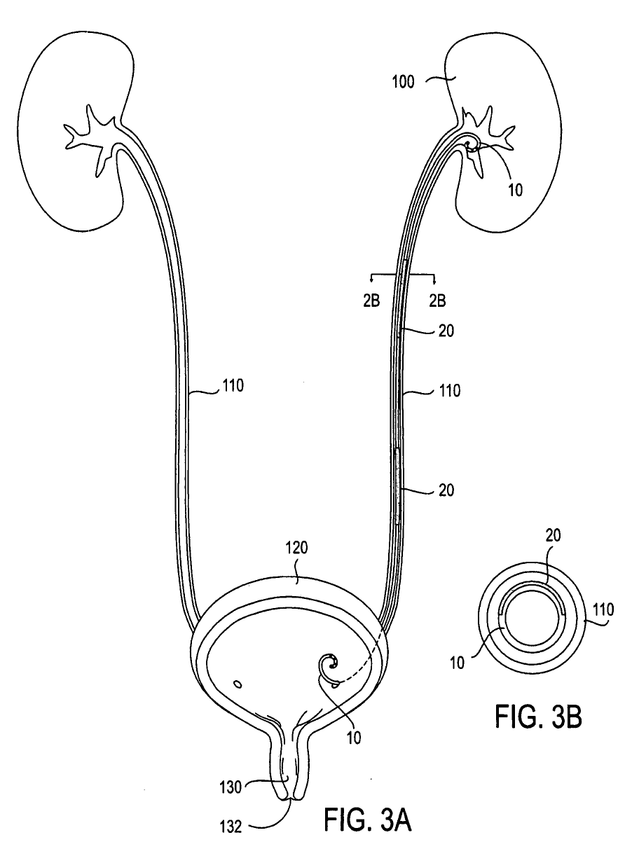

Embodiments of the invention include any device that dwells, or partly dwells in the urinary tract; the urinary tract extends from the kidney, through the ureters, the bladder, the urethra, and ends at the urethral opening. FIGS. 2 and 3 depict a schematic view of the urinary tract common to both males and females; the sexes differ in the length of the urerthra and in aspects of the surrounding urogenital organ system, but for general purposes, the sexually non-specific views of the urinary tract as provided by FIGS. 2 and 3 offer an appropriate orientation. Devices that may be inserted and dwell in the urinary tract may be located anywhere with the urinary tract, and may extend externally from the urinary through the urethral opening, as is the case with catheters, such as a Foley catheter. Many devices may be used in both sexes, but some devices are typically specific to one sex. Thus, exemplary devices, collectively and without intent to provide an exhaustive list, may include any of a ureteral stent, an upper urinary tract stent, (e.g., a double J stent), a lower urinary tract stent, a catheter (e.g., a Foley catheter), a urethral prosthesis, a urethral insert, an artificial urinary sphincter, a nephrostomy tube, a suprapubic tube, a urethral plug (e.g., Femsoft®) or any device dwelling within the urinary tract. All these listed devices currently exist as conventional devices, and all are included as embodiments of the currently described invention upon incorporation of—or upon being fitted with an embodiment of a motion-inducing actuator. Alternatively, or additionally, a range of energy emitting devices are available that can convey energy, such as ultrasonic energy, directly to a device that results in movement of the device. In some of these particular embodiments, the device may include physical features either integral to the device, or connected to it, that facilitate transduction of received energy into movement. In another aspect, all such just-detailed devices, when so-fitted or so-adapted, may be used by the methods described herein for inducing motion in such devices.

The description now turns to a consideration of movement of embodiments of the inventive devise and system, and methods by which movement is induced. By methods of the invention, the movement of the device, as imparted by a movement-inducing actuator, or as in some embodiments, as directly induced by the reception of wirelessly transmitted energy by the device, such as sonic or ultrasonic energy, may be broadly characterized as movement either in relation to the position of the device with respect to the urinary tract in which the device resides, or the movement may be within the device, such as by deforming or vibrating the device. Translational movement of the device with respect to the urinary tract location may occur along any of the three spatial axes in which the device can freely move. Additionally, or alternatively, device movement can be complex, combining movements along any of the axes. Thus, to the extent that movement is allowed by the constraints of the placement of the device within the urinary tract, induced movement may, by way example, move the device longitudinally (proximally or distally) within the urinary tract, or rotationally within the urinary tract.

Additionally, induced movement of a device may include vibration or deformation of the device, and this vibratory movement within the device may further occur in concert with the previously described translational movement of the device within the urinary tract. Vibrational or deformational movement may occur within the device, it may occur along any spatial dimension defined by the axes of the device, it may be complex, and it may occur with multiples modes, and it may include resonance. Vibration may manifest as waves in three dimensional form, moving through the device, in any direction. Further, vibration may manifest in the form of two dimensional surface area distortion or reconfiguration, with transient expansion and contraction of localized areas. In such a case, if an area were to be marked by reference lines (see FIG. 20) circumscribing a given surface area at rest, for example, distortion would show the lines to transiently circumscribe areas both larger and smaller than the referenced area at rest. Any of these recited forms of movement may be understood as being disruptive of the adherence of materials to the surface of an indwelling urinary tract device.

Movement of a device may, in brief, be understood in the context of the physics of rigid body translations and deformations. Thus, the “moving” of a device may include any of moving translationally with resonance along any of linear or rotational degree of freedom, moving translationally without resonance along any of linear or rotational degree of freedom, or moving deformationally with modes of any order, along any of linear or rotational degrees of freedom, or any combination of translational and deformational movement.

Movements of the device, whether translational within the urinary tract or vibrational or deformational of the device itself, may further be characterized variously as being regular, irregular, periodic, or random. Motion may vary in terms of magnitude or amplitude, frequency, and periodicity. Occurrence of movement may be episodic, or it may be continuous over a period of time. Occurrence of movement may be set to run on a schedule, whether defined by minutes, hours, days, weeks, or months. To an extent, some of these variables are a function of the dimensions of the device and physical constraints imposed on the device. All these variables are further a function of—and controllable by the form and rate of energy being conveyed to the device by an energy source, as variously conveyed through an energy receiver, an actuator, or directly to the device itself. These movement variables and parameters are well known in the art. Further well known are the hardware and software of control systems, feedback loops, memory chips and programmable chips, all of which are included as embodiments of a larger system that comprises the inventive device. In some embodiments, such as those that utilize wireless transmission of energy to a device, control systems are typically external. In other embodiments, where energy sources are attached to—or implanted in the body, the energy sources and actuator may comprise integrated chips that control the function of the device. Further, the methods of the present invention draw from these known systems and methods of controlling parameters, and apply them to the inventive producing of motion in implanted medical devices, such as the indwelling urinary devices described herein.

The description now turns to further consideration of various embodiments of the material deposition-counter urinary tract device with regard to adaptations that support movement. In some embodiments, the urinary tract device includes a motion-inducing actuator, as shown in the embodiments represented by FIGS. 1A-1D. Other embodiments, however, lack a distinct actuator and energy receiver, as described earlier in the context of FIG. 1E. Devices of either type, those with an actuator and those without an actuator may by particularly adapted to support or enhance movement of the device in response to receiving energy input. An example of such is an embodiment where energy is provided sonically, as for example by an external ultrasound emitter that is placed on or near the patient's body, and which is directed toward the device. In other embodiments lacking a distinct actuator, the device may include a portion that is particularly configured to receive and focus sonic energy, or simply to receive more energy than the device Would receive absent such portion. The energy focusing portion or element then moves with commensurately greater force or resonance, and then imparts that movement to other portions of the structure.

In other embodiments, the device as a whole may be configured to optimize or focus received energy. In embodiments such as these, where the device or a portion thereof is configured in a form to focus incoming energy, such as ultrasound, such focus may facilitate efficient reception and transduction of energy into resonance. In other embodiments where a distinct actuator is included with the device, the device and the actuator may be configured so as to optimally couple with each other. Also, physical features of shape or composition that support movement in devices that lack actuators, may be included in actuator-containing devices toward the same advantage. Further, while ultrasound is used as an example of applied wireless energy, energy directed to the device through an actuator may also arrive by a physical connection. Resonance is an advantageous response to incoming energy as it transduces incoming energy into vibrational movement with high efficiency.

Embodiments that focus or enhance a movement or vibrational response to incoming wireless energy may include variations in materials or shapes of conventional devices beyond or in addition to differences associated with the inclusion of an actuator per se. A typical form of incoming energy appropriate for these embodiments is ultrasound, as transmitted from an external probe. As an example of an inventive variation of a conventional device, the composition of plastics that comprise a Foley catheter, or a portion of a Foley catheter, may be varied so as to have an elastic modulus different (typically higher, but in some embodiments, lower) than that of conventional devices, such that the catheter or portion thereof which will propagate vibration at a commensurately greater efficiency. In another example, the plastic of a Double-J stent may be formed with walls that vary in thickness periodically along the length of the stent, the varied periodic pattern being calibrated so as to support a resonant standing wave (see FIG. 24 and further description below). In still other embodiments, as described further below, discrete energy focusing elements may be attached or integrated into a device that is otherwise substantially conventional in composition or form (see FIG. 25 and further description below).

The description now considers material deposition on surfaces and on devices that may not be tubular in form. Embodiments of the material deposition-countering device may counter the deposition of material on any surface of the device, including external surface and internal surfaces. Typical embodiments of the device are generally tubular in form, with a wall circumscribing a lumen, and circular or approximately circular in cross section. The cross sectional shape of such devices may vary, of course, according to the intended purpose of the device, and some portions of the device need not be tubular in form. Further, some urinary tract device embodiments may not be designed for the purpose of conveying fluid flow at all. Examples of such embodiments may include a wire or member of any kind, or a drug-eluting body; such devices comprise surfaces but may lack a lumen. The external surface of the tubular form of a device is adjacent to the wall of the portion of the urinary tract within which the device is residing, and the internal surface of the device circumscribes the lumen of the device through which urine flows. Materials deposited on the external surface of a device, by virtue of the close proximity of that surface to the inner surface of the urinary tract, are particularly vulnerable to disruption and dislodging by any kind of movement, translational, rotational, or vibratory, as provided by embodiments of the invention.

The description now further considers particulars of the materials that accumulate in devices in the urinary tract, which deposition is countered by embodiments of the inventive devise and method. In some embodiments, the device counters the deposition of material that includes a precipitate of one or more solutes included in the urine of the patient. Such precipitated solutes may include any one or more of calcium oxalate monohydrate, calcium oxalate dihydrate, calcium phosphate, magnesium ammonium phospate, struvite, hydroxyapatite, carbonate apatite, brushite, or uric acid. Precipitated solute in the form of stone or deposition typically commonly contains more than one of these species. Further, the composition of precipitated or deposited particulate matter varies according to the metabolic and dietary particulars of the individual, and accordingly, stones or deposition may contain other materials as well.

While material that precipitates in the urinary tract in the absence of a foreign nidus such as a device generally takes the form of a discrete stone, when being deposited on a device, it may take less discrete forms, for example taking the form of amorphous plaque. In other embodiments, the deposition-countered material may be of thrombotic origin. In other embodiments, the deposition-countered material may include cellular matter, such as whole cells, cell fragments, or cell-derived debris. In still other embodiments, the deposition-countered material may include a biologically-derived film, the film derived either from the patient or from an organism that has infected the patient. Finally, deposited material may include any of such materials, whether mineral or organic, in any proportion.

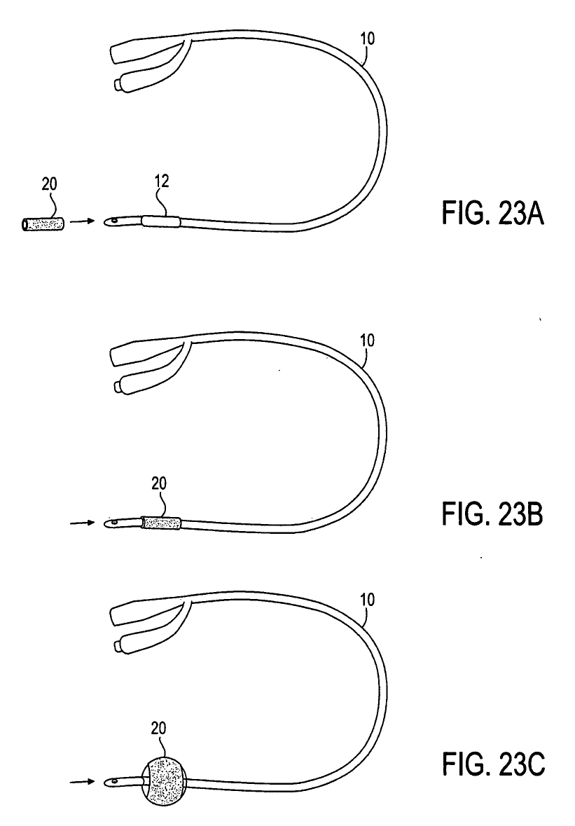

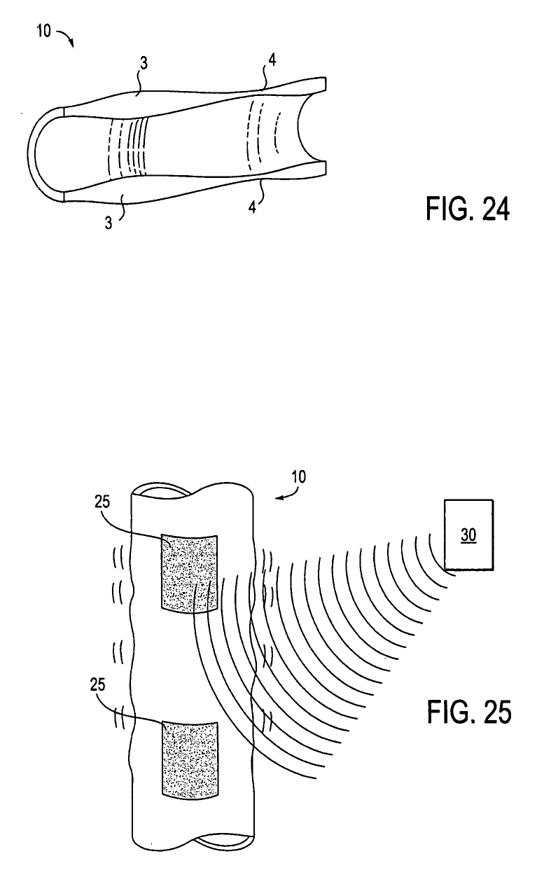

The deposition of material as described above may be countered or counteracted by embodiments of the device, as practice by embodiments of the method of using the device. Counteracting deposition may variously include impeding the deposition of material to any degree at all, it may include completely preventing deposition of material, and it may include any degree of impeding deposition between these two extremes. Countering deposition may further include removing or diminishing some portion of material that has already been deposited. This aspect of the method, where extant deposition is diminished, may be understood to occur in an embodiment where a device is fitted with a motion-inducing actuator after a device has been inserted or implanted into the urinary tract. Such an embodiment is depicted in FIGS. 22 and 23. Another case in which extant deposited material may be removed is in an embodiment where the device is induced to move as a whole by application of energy such as sonic or ultrasonic energy after such deposition has occurred, such as the embodiment depicted in the block diagram of FIG. 1e.

The description now turns to a consideration of motion-inducing actuators and energy sources included in the invention. Motion-inducing actuators 20, as applied to the structure of urinary tract devices 10 throughout this application may be any suitable device or element that can transduce an amount of energy received into movement of the device. Examples include any of a motor, a mechanical transducer, a piezoelectric element, a shape memory polymer, an electroactive polymer, a shape memory alloy (such as Nitinol, for example), a miniature actuator, an electromechanical actuator, or an electromagnetic actuator. Piezoelectric elements based on crystals that change shape in response to an applied voltage represent a typical embodiment, and offer the advantage of being easily shaped into an appropriate form, easily integrated into devices, for having no macroscopic moving parts, and for having a low physical profile. Accordingly, piezoelectric elements are suggested as actuators in numerous exemplary embodiments shown and described herein, although other types of actuators may serve equally as well. Various mechanical transducers are described and depicted herein as well, such as the embodiments shown in FIGS. 14-16, where the transducers capture the kinetic energy of flowing urine, and direct it moving the wall of the device. In some embodiments, typically those that transduce electromagnetic energy, the actuator may include integrated chips that control or mediate the transduction according to a stored program; and in some embodiments the chips which may further be reprogrammed wirelessly by an external programming device.

Energy sources 30, as applied to the inventive device described herein may by of any suitable type, physically connected or wirelessly connected to a motion-inducing actuator 20, or in some cases in wireless communication directly with a device such as that shown in FIG. 1e, where the device, in effect, acts as its own energy receiver and motion actuator. Wireless forms of energy source may include, for example, any of an inductive coil, a microwave emitter, a radiofrequency (RF) emitter, or a sonic emitter, such as an ultrasound probe. Energy sources that are typically physically connected to an actuator or device include any suitable type of battery or capacitor, and in typical embodiments would be implanted in the body, although some embodiments include external energy sources physically connected to a device by a lead. Further, in some embodiments, biological energy may be harnessed as a source, by way of thermal, electrochemical, or kinetic processes. In the embodiments shown in FIGS. 14-16, for example and as described further below, biological energy in the form of fluid flow provides energy. Embodiments of energy sources are typically under the control of electronic hardware and software that determine operational parameters under a stored program such as, for example, when to emit energy, at what level, and how to respond to incoming commands or feedback information. In some embodiments, typically where the energy source is external these mechanisms are external; in other embodiments, where the energy source is attached to the body or implanted therein, the controls are integrated into the device in the form of memory chips and programmable chips.

The description now turns to a consideration of exemplary embodiments, as depicted in FIGS. 2-27. FIG. 2A and 2B schematically represent a urinary tract, including the kidneys 100 which generate urine that flows through ureters 110 into the bladder 130 where it is held until released into the urethra 130, and exits the body through the urethral opening 132. FIG. 2 shows an example of a conventional indwelling urinary tract device 9 in the form of a Double J stent, with one J-shaped end within a kidney 100, the lengthy tubular portion extending through the length of a ureter 110, and the other J-shaped end disposed in the bladder 130. Deposited or precipitated material 50 in the form of kidney stones is shown both in the kidney and in a ureter. Additionally, the stone material 50 is shown in form of encrustation within the length of the device 9 in FIG. 2A, as well as in the cross sectional views of FIGS. 2B and 2C. The cross sectional view of FIG. 2B shows encrustation on the external surface of device 9, occupying the space between the device 9 and the inner wall of ureter 110. Encrustation in this form plainly can be the cause of pain and irritation and damage to the ureter, as well as being problematic for removal, as well as providing a surface receptive to bacterial colonization. The cross sectional view of FIG. 2C shows encrustation on the internal surface of device 9, occupying lumen 15 of the device 9, partially occluding it. In some cases, such material may completely occlude the lumen 15.

FIG. 3A and 3B show a the same view of a urinary tract as depicted in FIG. 2 with an indwelling device, but in this case the urinary tract device 10 is an embodiment of the present invention. In this embodiment, the device 10 can be seen to have an actuator element 20 integrated in the form of discontinuous strips along the length of the central tubular portion of the device 10. FIGS. 3A and 3B (a cross section of the device within the ureter) depict a urinary tract that is free of any encrustational build up 50 as is painfully evident in FIGS. 2A-C, showing a urinary tract fitted with a conventional device. This form of a motion-inducing actuator is but one embodiment, further embodiments are described below. This embodiment may be, for example a piezoelectric element which moves in response to energy input. Not shown in this figure, although it may be considered present, is a form of energy input, wireless, for example, and an energy receiver, such as an antenna, which transfers energy to the piezoelectric strip. FIGS. 3A and 3B thus represent a device that has been occupying a urinary tract that would have otherwise accumulated material (as shown in FIGS. 2A-C) were it not for the movement of the device which has impeded or, as in this case, entirely prevented such material build up.

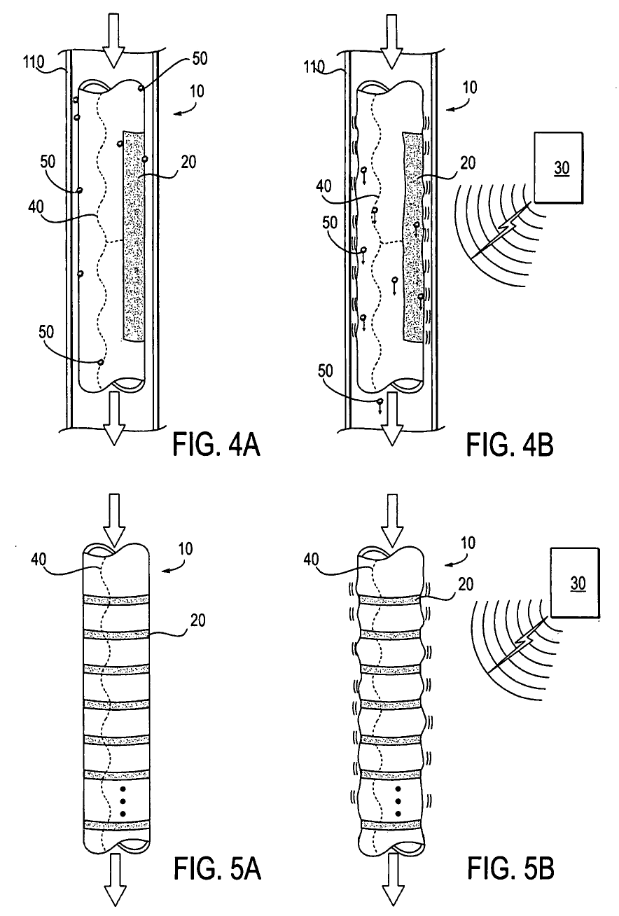

FIGS. 4-7 depict various embodiments of the material deposition countering device both at rest and while moving in response to motion-induction by an actuator 20. FIG. 4A shows a portion of a device 10 residing in a portion of a ureter 110, which is representative of any portion of the urinary tract. The direction of urine flow is downward, as depicted by arrows, and as is the case for all of FIGS. 4-7. Urine flow may participate in the dislodging of particulate material from the surface of devices and the internal surface of the urinary tract, flushing the surface and sweeping such particulate out of the body. FIG. 4A also shows an actuator in the form of a discrete longitudinally configured strip 20, a piezoelectric element, for example, and an energy receiver 40, such as an antenna, operably connected to the acutator 20. Also apparent are various pieces of mineral or stone 50, either free floating or attached to the device or to the wall of the ureter 110. FIG. 4B shows the same device, moments later, for example, as it is moving in response to motion induction by the actuator 20, the actuator receiving energy wireless from energy source 30. Movement is shown by small movement lines along the wall of device 10. In response to such movement, particulate material 50 has become dislodged from attachment sites on the device and on inner aspect of the urinary tract 110, and is being swept downstream by urine flow.

FIGS. 5A and 5B show a device 10 similar to that shown in FIG. 4, except that actuator 20 is in the form of a series of circumferential strips (the series of black dots indicates the continuation of such strips) that are connected to an energy receiver or antenna 40, and the device is shown outside the context of a portion of the urinary tract. FIG. 5B shows the device 10 in motion as motion is being induced by the actuator 20, as it is being powered by energy source 30, as being delivered through energy receiver 40.

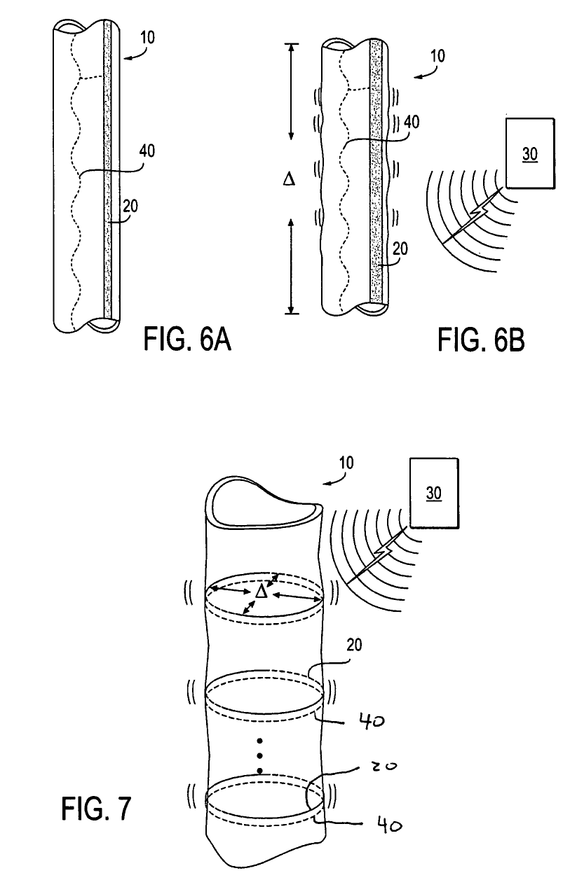

FIGS. 6A and 6B show views of a device 10 similar to that of FIG. 5A and 5B, with an actuator 20 in the form of a continuous longitudinal strip 20 that is connected at intervals to energy receiver or antenna 40. FIG. 6B shows the device as motion is being induced by the actuator 20, as it receives energy from the energy source 30 through the intervention of receiver or antenna 40. The motion, in this case the motion takes the form of longitudinal compression and expansion, as indicated by arrows. This motion can be readily understood as one that distorts the surface of the device through transient expansion and contraction

FIG. 7 shows an embodiment of the device 10 in which the actuator 20 and the energy receiver 40 both take the form of a series of circumferential rings. Movement as indicated by arrows, and as induced by the actuators 20, can be seen to involve expansion and contraction of the device radially at the site of the actuators. Such from of movement, like that seen in FIG. 6B also entails expansion and contraction of surface area, and such distortions have the effect of disrupting and preventing attachment of particles to the surfaces of the device 10.

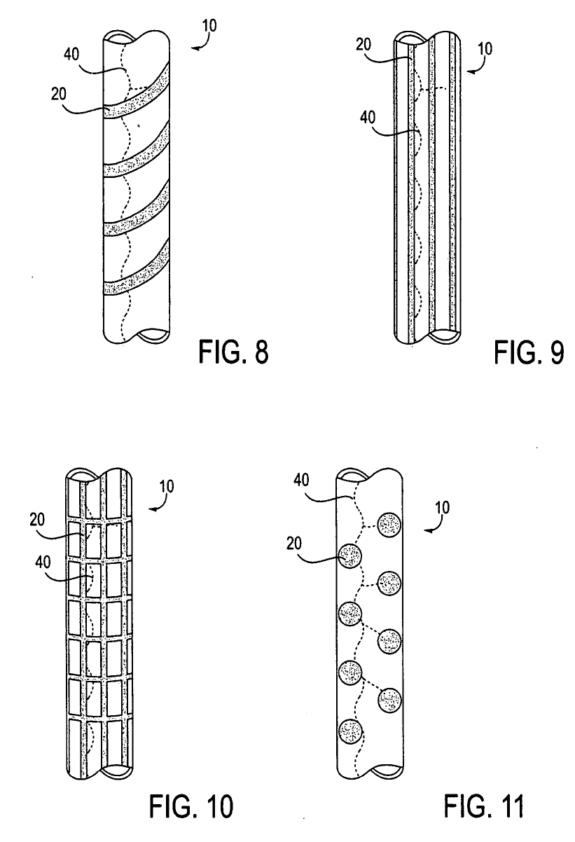

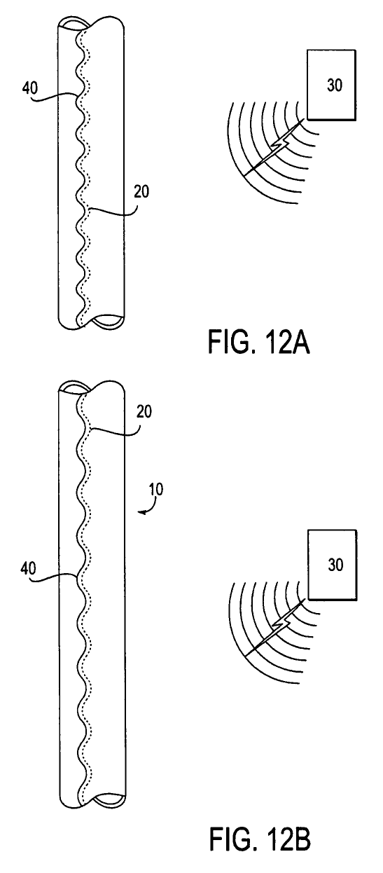

FIGS. 8 -12 depict further embodiments of device 10 which vary in the configured form of the actuator 20. FIG. 8 depicts the actuator 20, a piezoelectric element for example, as a continuous cirumferential coil that is connected to an antenna 40. FIG. 9 depicts the actuator 20, a piezoelectric element for example, as a series of parallel continuous strips connected to an antenna 40. FIG. 10 depicts the actuator 20, a piezoelectric element for example, as a latticework that is connected to an antenna 40. FIG. 11 depicts the actuator 20, a piezoelectric element for example, as distribution of discrete sites, each site being connected to an antenna 40. FIG. 12A and 12b depict the actuator 20, a piezoelectric element for example, longitudinally-oriented wave-shaped coil. The two FIGS. 12A and 12B show the device at a state of maximal contraction and maximal extension, respectively. The wave-shaped actuator 20 in the two figures and emphasizes the states of contraction (as in FIG. 12A) and extension (as in FIG. 12B); and further provides another example of disruptive surface area distortion.

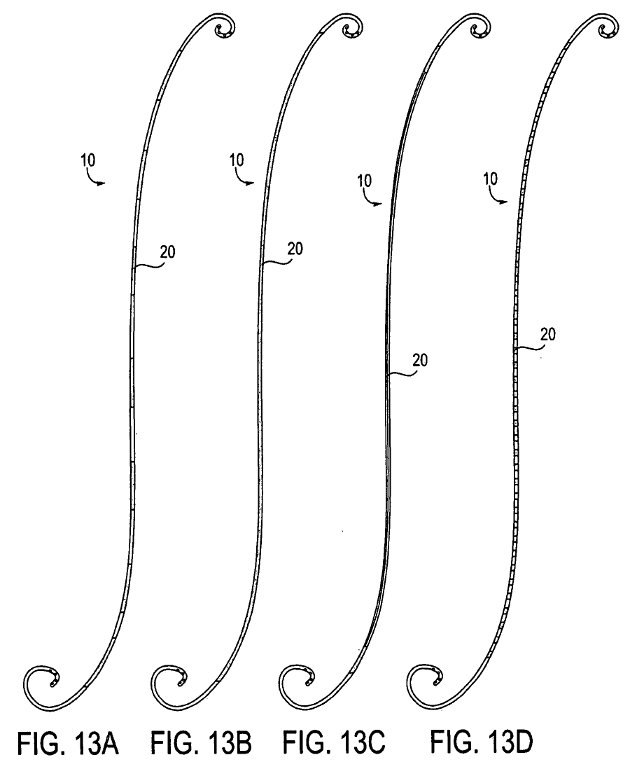

FIGS. 13A-13D show various embodiments of a specific type of indwelling urinary tract device 10, a Double-J stent, which is designed to occupy the upper urinary tract as seen in FIGS. 2 and 3. These embodiments vary in terms of the form of the motion-inducing actuator 20. FIG. 13A shows the actuator 20, a piezoelectric element, for example, in the form of discontinuous strips, occupying all or a portion of the circumference (an antenna or energy receiving element is not shown in this set of figures). FIG. 13B shows the actuators 20 distributed substantially throughout the device 10, as layered, interleaved, intermingled, or woven elements. Discrete actuating and energy receiving elements such as those depicted in this embodiment, or in any other embodiment described herein, may be formed of—or encased in biocompatible materials. FIG. 13C shows the actuator 20 as a long continuous strip. FIG. 13D shows the actuator 20 portion as a continuous spiral substantially along the full length of the device 10.

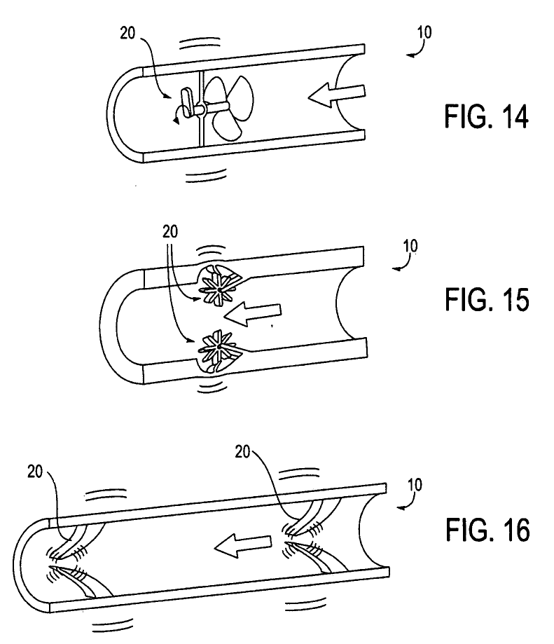

FIGS. 14 -16 show embodiments of the device in which the actuator 20 assumes the form of a transducer that converts the kinetic energy of periodic urine flow into kinetic energy that moves or vibrates the device. FIG. 14 shows an actuator 20 as a miniature propeller disposed in the path of urine flow with an eccentric counterweight; the propeller assembly is connected to the wall of the device, the eccentric counterweight drives movement of the wall of the device. FIG. 15 shows an actuator 20 embodiment comprising two miniature waterwheels attached to the wall of a device; as they turn under the force of urine flow, the wheels flick a barb attached to the inner wall surface, thereby imparting a motion or vibration to the wall of the device 10. FIG. 16 shows an actuator 20 embodiment comprising flaps attached to inner surface of the wall of device 10. The actuating flaps 20 have a centrally-oriented bias into the lumen 15 of the device, and are pushed aside by the urine flow. The tension between the inward bias and deflecting pressure of urine flow creates movement that is transferred to the wall of the device.

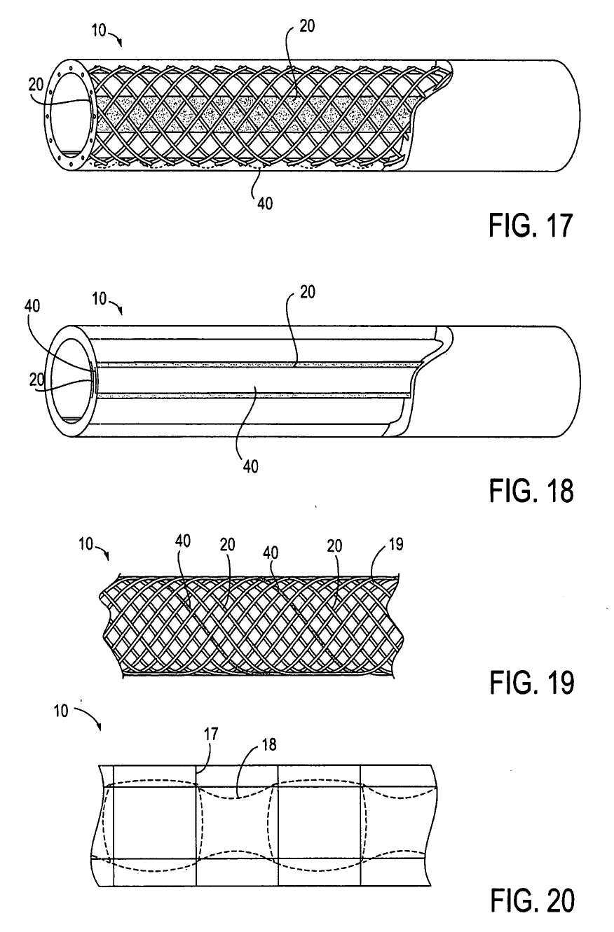

FIG. 17 depicts another embodiment of the device 10 with an antenna 40 and motion-actuating elements 20 integrated into a structure that is otherwise substantially formed from a polymer or any elastomeric material. This device differs from those depicted in previous figures in terms of the complexity and the degree of integration of structural elements and the functional elements of the invention, such as a motion-inducing actuator and antennae. The woven metallic components in this embodiment serve the purpose of strengthening the overall structure, but further include the actuator 20 and antenna 40 elements as functional elements.

FIG. 18 is a perspective and partially cut-away view a portion of a device with an antenna 40 and a motion-actuating elements 20 integrated into a structure that is otherwise substantially formed from polymer or other suitable embedding material. In this embodiment the actuator 20 and the antenna 40 do not comprise a structurally-supporting mesh as in FIG. 17, but are nevertheless integrated into the primary structural material of the device; in this particular embodiment, the actuator 20, a piezoelectric element, for example, and the antenna 40 are adjacent and layered over each other. In embodiments of this type, the actuator and the antenna may assume various forms, as depicted variously in FIGS. 3-13, and may assume any form which allows the energy receiver to receive and transfer energy, which allows the actuator to effect motion in the structure, and which allows an operable connection between the two elements, the energy receiver and the actuator. Such forms may include the elements being interwoven, one layered over the other, elements being adjacent to each other, alternating, interspersed, or being laid out in any pattern, regular or irregular, that is consistent with functional interaction.

FIG. 19 provides a perspective view of a device 10 in the form of a bare metal wire stent, with antenna 40 and motion-inducing actuator 20 elements integrated into the woven wire. In this embodiment, the structure of device 10 is formed substantially by metal wires or struts 19, and does not include an encasement, housing, or overall covering by a structure substantially formed of polymer or elastomeric material, such as those depicted in FIGS. 17 and 18. Metals comprising this embodiment include those known in the art to be biocompatible; further or alternatively, structural metal may be coated with a layer or layers of biocompatible metal or polymer.

FIG. 20 shows a face-on flattened planar view of a portion of a wall of a device 10 with solid reference lines 17 depicting the surface area in a neutral state and dotted reference lines 18 depicting a stop-action view of the surface area when the device 10 is vibrating due to motion actuation, and the reference lines depicting the distortion of the surface area, with some sectors expanded, and others contracted. This form of movement, within the wall of the device 10 structure is a type of movement common to many embodiments described herein. This type of movement is one that impedes, discourages, prevents, or disrupts particulate material from association or binding with the surface, and accumulation thereon. It can be understood, by way of an exemplary analogy, that it is unlikely that paint would stick to a balloon that is inflating and deflating.

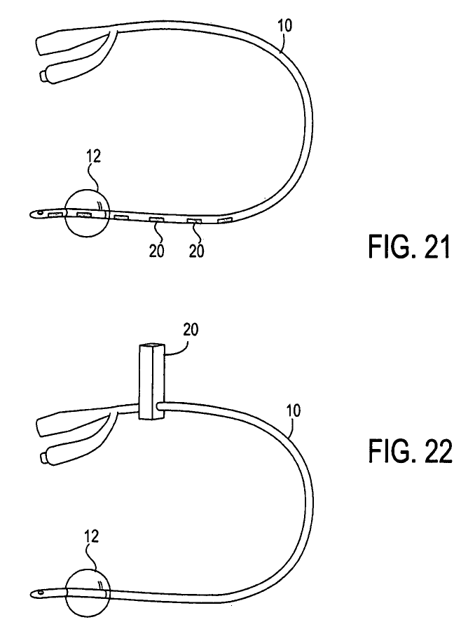

FIGS. 21-23 show embodiments of the material deposition countering device 10 in the form of a Foley catheter which is used very commonly in hospitals and nursing facilities, which dwells in the lower urinary tract and extends externally from the urethral opening. FIG. 21 shows an embodiment of a Foley Catheter with a motion-inducing actuator 20 integrated into the device, in the same manner as that seen in the double-J stent of FIG. 12A, for example. FIG. 22 shows an embodiment of a device 10 in the form of a Foley catheter, with a motion-inducing actuator 20 attached and operably connected to the device, not integrated into it. In this embodiment, the actuator is shown to be attached to the Foley catheter on a portion of that is outside the body. In other embodiments, a motion-inducing actuator 20 may also be attached to the device at a site internal to the body, as for example, depicted in FIGS. 23A-C. Of note with regard to the base structure of this device is that in some embodiments, without being combined with the actuator, the device may be otherwise be conventional. In embodiments such as these, the devices depicted in FIGS. 21-23 are but one example, the invention comprises the combination of the base structure with the actuator, the two components being operably connected.

FIGS. 23A-23C show an embodiment of a device 10 in the form of a Foley catheter, a partially indwelling urinary tract device, with a motion-inducing actuator 20 in the form of a sleeve fitted over the balloon end 12 of the catheter. FIG. 23A shows the Foley with the balloon portion 12 deflated, the actuator sleeve prior to be fitted over the balloon, FIG. 23B shows the sleeve fitted over the balloon portion, and FIG. 23C shows the sleeve 20 over the balloon portion after the balloon has been inflated.

Energy may be provided to the motion-inducing actuator from a source either within the body, or from outside the body. The embodiments depicted in FIGS. 14-16, for example are sources from inside the body in the form of energy being derived from a physiological process, in this case, the process of urine flow. In other embodiments, other biological processes may be harnessed as an energy source, such as other forms of kinetic activity, chemical processes, electrochemical processes, or thermal gradient processes. In other embodiments, an energy source such as a battery or capacitor, typically rechargeable, may be implanted and operably connected to the actuator. Implanted energy sources may fully implanted, or partially-implanted, or temporarily- or short-term implanted. In other embodiments, the energy source may be external to the body, but attached to it either directly, or it may be attached by being including within an article that is worn, such as a harness, belt, or an article of clothing by approaches well known in the art. In other embodiments, the energy source is unattached to the body, and energy is provided to the actuator 20 or the device 10 directly, as in the case of ultrasonic activation, by the source and the patient being placed in mutual proximity. An example is that of a handheld unit which the patient holds at an appropriate position relative to the body, and then activates. Some embodiments of an external energy source are not handheld, by virtue of being too large to be conveniently held, or by being configured for another form of use. One such example is where the energy source is included in a bed on which the patient lays.

FIG. 24 depicts an embodiment of a device that has been adapted to optimize the receipt of energy and transduce it into movement. In this embodiment, the wall of the device 10 has been formed of undulating thickness along its length, more specifically of uniform thickness at any circumscribed ring, but of varying thickness of a regular periodicity longitudinally. FIG. 24 thus shows a cutaway view of a tubular portion of a device 10 that includes thickened portions 3 of wall and thin portions 4 of a wall. Such variation in thickness in a periodic form is to enhance resonance by supporting a standing wave of the same length. Energy may be provided to the device by an energy source either as mediated by an actuator, as previously described, or directly by an energy source without the mediation of an actuator, as has also been described.

FIG. 25 depicts an embodiment of a device 10 that is adapted to optimize the receipt of energy transmitted from energy source 30 without the mediation of an actuator. The device has been fitted with an energy focusing or targeting element 25 that is particularly efficient at receiving wireless energy and moving in response. In various embodiments, the element 25 may incorporated into the device 10, or fixedly applied to it. In embodiments where the energy targeting element is applied to a device, in the absence of such attachment, the device may otherwise be a conventional device. Thus, of note with regard to the base structure of this device is that in some embodiments, without being combined with the targeting or focusing element, the base device may otherwise be conventional. In embodiments such as these, of which the one depicted in FIGS. 25 is but one example, the invention comprises the combination of the base device with the focusing element, the two components being connected such that movement of the focusing element is imparted to the structure as a whole.

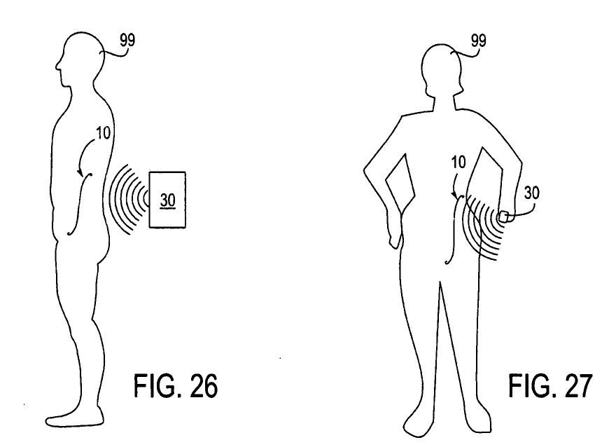

FIGS. 26 and 27 provide examples of an external energy source being applied to a device in situ. FIG. 26 shows a patient 99 with an indwelling device 10 in the form of a Double J stent, the device being powered by an external energy source 30. FIG. 27 shows a patient 99 with an indwelling device 10 in the form of a double J stent, the device being powered by a handheld external energy source 30. The Double-J stent 10 may include a motion-inducing actuator (not shown) of any suitable form, such as those exemplified in FIGS. 12A-12D. In still other embodiments, the Double-J stent may not have a distinct actuator, but rather be configured so as to focus energy and optimize resonance thereby, as described above exemplified in an embodiment shown in FIG. 24. Alternatively, the Double-J stent could be fitted with an energy focusing element, as described above and exemplified in an embodiment shown in FIG. 25, that imparts resonance to the stent as a whole. An external energy source may be of any suitable type, including for example, an inductive coil, a microwave emitter, a radiofrequency emitter, or a sonic emitter, such as an ultrasound emitter. In embodiments of the device without an actuator, a typical external energy source is an ultrasound emitter.

The system and method described herein in the context of devices dwelling in the urinary tract may be also applied to any other indwelling or implanted device that is subject to receiving material deposition, and accordingly, are included as embodiments of this invention. Any such device adapted to at least partially dwell in the body may include a structure operably coupled to an actuator such that the actuator can induce motion in or of the structure, or alternatively, the device may be adapted to move in response to receiving or absorbing energy directed to it, as has been described above, particularly with reference to energy-focusing features or elements that may be adapted or configured in otherwise conventional devices. Further, as mentioned previously, embodiments need not include a lumen as does the urinary tract embodiment. Still further, in broader aspect, absorbing of energy by embodiments of the invention may include generation of heat in concert with movement, or alternatively, instead of movement.

Thus various embodiments of the inventive device may be those placed in any suitable space within the body, as for example, any space that accommodates a conventional medical device. For example, embodiments of the device may dwell in a body lumen, such as an auditory canal, a blood vessel, the heart, a bile duct, a lymphatic vessel, a salivary gland such as a parotid gland, a sinus, or a lacrimal duct, or a pulmonary lumen. An example of an embodiment of the invention thus may include a cardiac stent, wherein movement of the stent may prevent clot formation, particularly in the early post-implant period. In other embodiments, the device may dwell in a hollow organ, for example a portion of the alimentary canal, including any portion of the upper gastrointestinal tract, the stomach, and the lower gastrointestinal tract, the latter portion including the small and large intestine. An exemplary embodiment of the inventive device and method in this context may include an espophageal stent, where energy absorbance may render a therapeutic scarring to treat reflux. Still other embodiments may dwell in a body cavity, such as the pleural cavity or the peritoneal cavity. An example of such a device is a peritoneal dialyis catheter, which is subject to clogging by accumulation of material on its external surface. Other embodiments, as for example leads or sensors, may dwell in the interstitial space of any tissue or organ. The useful lifespan and effective operation of devices such as these are known to be frequently compromised by accumulation of material or adherence of cells and cellular material, and accordingly may benefit by disruptive movement.

Devices situated in these various locations in the body, such as a lumen, cavity, or hollow organ, will be adapted in size and shape appropriate for their designated function. As mentioned in the embodiments described herein that dwell in the urinary tract, these devices need not include a lumen. In fact, the only physical qualification is that the device comprise a surface, and for the inventive aspect of the device to be useful, the only qualification is that the surface be subject to a problematic deposition of material. Further, the environment in which embodiments of the device dwell need not be one that is bathed in or flushed by flowing fluid. The peritoneal cavity, for example, may contain fluid, but the fluid has no directional mass flow. The auditory canal, for another example, contains waxy secretion, but it is not liquid, and doesn't flow as a fluid. In embodiments of the device that are situated in environments such as these, the benefit of translational or deformational movement may be to prevent or impede the build up of environmental material, or the benefit may be to render the material into a state, such as being less dense or more loosely attached, for example, that provides functional benefit for the device, or renders the material into a state more easily removed by other means.

While the inventive device and method, as illustrated by the describe embodiments have been described in some detail by way of illustration, such illustration is for purposes of clarity of understanding only. Various terms have been used in the description to convey an understanding of the invention; it will be understood that the meaning of these various terms extends to common linguistic or grammatical variations or forms thereof. It will also be understood that when terminology referring to devices or equipment has used trade names, brand names, or common names, that these names are provided as contemporary examples, and the invention is not limited by such literal scope. Terminology that is introduced at a later date that may be reasonably understood as a derivative of a contemporary term or designating of a subset of objects embraced by a contemporary term will be understood as having been described by the now contemporary terminology. Further, while some theoretical considerations have been advanced in furtherance of providing an understanding of an invention, the claims to the invention are not bound by such theory. Further, it should be understood that the invention is not limited to the embodiments that have been set forth for purposes of exemplification, but is to be defined only by a fair reading of claims that are appended to the patent application, including the full range of equivalency to which each element thereof is entitled.

What is claimed is:

1. A material deposition-countering urinary tract device comprising:
a structure adapted to dwell at least partially in the urinary tract; and
an actuator operably coupled to the structure such that actuator activation induces motion of the structure.

2. The device of claim 1 wherein the structure is any of a ureteral stent, an upper urinary tract stent, a lower urinary tract stent, a catheter, a urethral prosthesis, a urethral insert, an artificial urinary sphincter, a nephrostomy tube, a suprapubic tube, a urethral plug, or any structure adapted to dwell within the urinary tract.

3. The device of claim 1 wherein the urinary tract comprises any one or more portions of a kidney, a ureter, a bladder, or a urethra.

4. The device of claim 1 wherein the device comprises a surface with an area, and wherein the induced motion comprises a transient reconfiguration of the surface area.

5. The device of claim 1 wherein the motion-inducing actuator is any of a motor, a mechanical transducer, a piezoelectric element, a shape memory polymer, an electroactive polymer, a shape memory alloy, a miniature actuator, an electromechanical actuator, or an electromagnetic actuator.

6. The device of claim 1 wherein the device is configured to focus received wireless energy.

7. The device of claim 6 wherein the motion-inducing actuator and the structure of the device are structurally integrated.

8. The device of claim 6 wherein the energy is ultrasonic energy.

9. The device of claim 1 wherein the motion-inducing actuator comprises a mechanical transducer powered by a physiological process.

10. The device of claim 9 wherein the physiological process comprises urine flow, and wherein the mechanical transducer is a fluid-flow energy transducer in the urine flow path.

11. The device of claim 1 further comprising an energy source to power the motion-inducing actuator.

12. The device of claim 11 wherein the energy source is physically connected to the actuator, the source comprising any of an energy storage device.

13. The device of claim 11 wherein the energy source is configured to deliver energy wirelessly.

14. The device of claim 13 wherein the wireless energy source is any of an inductive coil, a microwave emitter, a radiofrequency emitter, or a sonic emitter.

15 The device of claim 13 wherein the energy source is external to the body.

16. The device of claim 15 wherein the energy source is attached to the patient.

17. The device of claim 15 wherein the energy source is unattached to the patient.

18. The device of claim 11 wherein the energy source is implanted in the body.

19. The device of claim 1 further comprising a wireless energy receiver configured to convey energy to the motion-inducing actuator.

20. The device of claim 19 wherein the wireless energy receiver is any of an antenna, a sonic transducer, or an electromagnetic-mechanical transducer.

21. The device of claim 1 wherein the deposition on the device is on any of an external surface of the device or an internal surface of the device.

22. The device of claim 1 wherein the deposition comprises a precipitate of at least one solute from urine.

23. The device of claim 22 wherein the precipitated solute from urine includes any one or more of calcium oxalate monohydrate, calcium oxalate dihydrate, calcium phosphate, magnesium ammonium phospate, struvite, hydroxyapatite, carbonate apatite, brushite, cystine, indinavir, triamterene, or uric acid.

24. The device of claim 1 wherein the deposition is of thrombotic origin.

25. The device of claim 1 wherein the deposition comprises cellular material, the material including any of whole cells, cell fragments, or cell-derived debris.

26. The device of claim 1 wherein the deposition comprises a biologically-derived film derived from any of the patient or an organism residing in the patient.

27. A material deposition-countering urinary tract system comprising:
a device adapted to dwell at least partially in the urinary tract and an energy source adapted to provide energy to move the device.

28. The system of claim 27 wherein the structure is any of a ureteral stent, an upper urinary tract stent, a lower urinary tract stent, a catheter, a urethral prosthesis, a urethral insert, an artificial urinary sphincter, a nephrostomy tube, a suprapubic tube, a urethral plug, or any structure adapted to dwell within the urinary tract.

29. The system of claim 27 wherein the urinary tract comprises any one or more portions of a kidney, a ureter, a bladder, or a urethra.

30. The system of claim 27 wherein the device comprises a surface with an area, and wherein the induced motion comprises a transient reconfiguration of the surface area.

31. The system of claim 27 wherein the device is adapted to focus received wireless energy.

32. The system of claim 31 wherein the received energy is ultrasonic energy.

33. The system of claim 31 wherein the adaptation comprises an energy focusing element attached to the device that moves in response to the energy, the element configured to impart movement to the device as a whole.

34. The system of claim 31 wherein the adaptation comprises an energy focusing element integral to the device that moves in response to the energy, the element configured to impart movement to the device as a whole.

35. The system of claim 31 wherein the adaptation comprises the device including a composition with an elastic modulus different than that of a conventional device.

36. The system of claim 31 wherein the adaptation comprises the device having a physical form that supports a resonant standing wave.

37. The system of claim 27 wherein the energy source is configured to provide energy wirelessly.

38. The system of claim 37 wherein the wireless energy source is any of an inductive coil, a microwave emitter, a radiofrequency emitter, or an ultrasonic emitter.

39. The system of claim 27 wherein the energy source is external to the body.

40. The system of claim 39 wherein the energy source is attached to the patient.

41. The system of claim 39 wherein the energy source is unattached to the patient.

42. The system of claim 27 wherein the deposition on the device is on any of an external surface of the device or an internal surface of the device.

43. The system of claim 27 wherein the deposition comprises a precipitate of at least one solute from urine.

44. The system of claim 43 wherein the precipitated solute from urine includes any one or more of calcium oxalate monohydrate, calcium oxalate dihydrate, calcium phosphate, magnesium ammonium phospate, struvite, hydroxyapatite, carbonate apatite, brushite, cystine, indinavir, triamterene, or uric acid.

45. The system of claim 27 wherein the deposition is of thrombotic origin.

46. The system of claim 27 wherein the deposition comprises cellular material, the material including any of whole cells, cell fragments, or cell-derived debris.

47. The system of claim 27 wherein the deposition comprises a biologically-derived film, derived from any of the patient or an organism residing in the patient.

48. A method of countering material deposition on a urinary tract system comprising a device adapted to dwell at least partially in the urinary tract and an energy source adapted to provide energy to move the device, the method comprising moving the device.

49. The method of claim 48 wherein the system further comprises an actuator operably coupled to the structure such that actuator activation induces motion of the device.

50. The method of claim 49 wherein the energy source is configured to convey energy to the motion-inducing actuator.

51. The method of claim 48 wherein the device comprises an energy focusing element that moves in response to the energy, the element configured to impart movement to the device as a whole.

52. The method of claim 48 wherein moving the device comprises moving it sufficiently to counter the presence of material on the device.

53. The method of claim 52 wherein countering material deposition comprises any of impeding material deposition, preventing material deposition, diminishing an extant deposition of material, or clearing a device occluded by deposited material such that urine may flow through the structure.

54. The method of claim 48, wherein moving comprises any of moving translationally with resonance along any of linear or rotational degree of freedom, moving translationally without resonance along any of linear or rotational degree of freedom, or moving deformationally with modes of any order, along any of linear or rotational degrees of freedom, or any combination of translational and deformational movement.

55. The method of claim 48 wherein moving the device sufficiently to counter the presence of material deposition comprises moving the device for a sufficient amount of time.

56. The method of claim 55 wherein a sufficient amount of time comprises any of sufficient total amount of time per day, a sufficient total amount of time per week, a sufficient amount of time per month, or a sufficient period of time during the residence of the device.

57. The method of claim 55 wherein moving the device occurs episodically, and wherein a sufficient period of time comprises moving the device for a sufficient amount of time per episode.

58. The method of claim 48 wherein moving the device sufficiently to counter the presence of material deposition comprises moving with sufficient power.

59. The method of claim 58 wherein moving the device with sufficient power comprises moving the device through a range of power.

60. The method of claim 48 wherein moving the device sufficiently to counter the presence of material deposition comprises moving the device at an effective vibrational frequency.

61. The method of claim 60 wherein moving the device at an effective vibrational frequency comprises moving the device through a range of frequencies.

62. A material deposition-countering bodily indwelling system comprising:
a device adapted to dwell at least partially in a space in the body and an energy source adapted to provide energy to move the device.

63. The system of claim 62 wherein the system further comprises an actuator operably coupled to the structure such that actuator activation induces motion of the device.

64. The system of claim 62 wherein the space in the body comprises a lumen.

65. The device of claim 64 wherein the lumen may comprise any of an auditory canal, a blood vessel, a heart, a bile duct, a salivary gland, a parotid gland, a sinus, or a lacrimal duct, or a pulmonary lumen.

66. The system of claim 62 wherein the space in the body comprises a cavity.

67. The device of claim 66 wherein the cavity may comprise any of a pleural cavity or a peritoneal cavity.

68. The system of claim 62 wherein the space in the body comprises an alimentary canal.

69. The system of claim 62 wherein the space in the body comprises an interstitial space.

70. A method of using the system of claim 62, the method comprising moving the device.

71. A material deposition-countering bodily indwelling system comprising:
a device adapted to dwell at least partially in a space in the body and an energy source adapted to provide energy to heat the device.

72. A method of using the system of claim 71, the method comprising heating the device.
